(19) United States
(12) Patent Application Publication
(10) Pub. No.: US 2004/0023229 A1
(43) Pub. Date: Feb. 5, 2004

(54) Direct detection of individual molecules

(76) Inventor: Rudolf Rigler, St-Sulpice (CH)

Correspondence Address: FULBRIGHT & JAWORSKI, LLP, 666 FIFTH AVE, NEW YORK, NY, 10103-3198, US

(21) Appl. No.: 10/240,788

(22) PCT Filed: May 11, 2001
(86) PCT No.: PCT/EP01/05408

(30) Foreign Application Priority Data
May 12, 2000 (DE) 100234232

(51) Int. Cl. C12Q 1/68; G01N 33/53

(52) U.S. Cl. 435/6; 435/7.1

(57) ABSTRACT

The invention relates to a method for directly detecting an analyte in a sample fluid and to an apparatus suitable therefor.

### DESCRIPTION

The invention relates to a method for directly detecting an analyte in a sample fluid and to an apparatus suitable therefor.

In diagnostic methods analytes are detected in biological samples, these analytes often being present only at a very low concentration. A direct detection of the analyte, however, is problematic, in particular for analyte concentrations in the range of ≦10⁻¹² mol/l, for example in the case of virus particles.

In order to detect a nucleic acid analyte at very low concentrations, it is possible to increase the number of the analyte molecules in the sample by amplification methods such as PCR or analogous methods to a concentration level which makes possible a detection by conventional methods such as, for example, gel electrophoresis or sequencing. However, such amplification methods are very time-consuming and have many sources of error, so that the appearance of false-positive or false-negative test results cannot be ruled out.

The European patent 0 679 251 describes a fluorescence correlation spectroscopy (FCS) method which represents a direct detection of individual analyte molecules. By means of FCS it is possible to detect a single or only a few fluorescent dye-labeled molecules in a small measuring volume of, for example, ≦10⁻¹⁴ 1. The measuring principle of FCS is based on exposing a small volume element of the sample fluid to a strong excitation light, for example of a laser, so that only those fluorescent molecules which are present in said measuring volume are excited. The emitted fluorescence light of this volume element is then projected onto a detector, for example a photomultiplier. A molecule located in the volume element leaves the volume element again according to its characteristic rate of diffusion after an average time which is, however, characteristic for the relevant molecule and can no longer be observed thereafter.

If the luminescence of one and the same molecule is then excited several times during its average stay in the measuring volume, a multiplicity of signals from said molecule can be recorded.

In order to reduce the measuring time which can be relatively long and depends on the rate of diffusion of the molecules involved, the European patent 0 679 251 describes various methods which can be used to concentrate in the measuring volume the molecules to be detected. In principle, these methods are based on preconcentrating the analyte to be detected using a directed electric field or else utilizing the different rates of diffusion of the molecules in the sample, owing to the different molecule size.

The German patent 195 08 366 describes an application of the FCS method to the direct detection of analytes in a sample. In this connection, a test solution containing a mixture of different short primers which have in each case an “antisense sequence” complementary to a section of a nucleic acid analyte and which are labeled with one or more dye molecules is provided. This test solution is mixed with the solution to be examined and the mixture is incubated to hybridize the primers with the nucleic acid strands to be detected. Then the target sequences in the incubated solution are identified by discriminating a few, preferably one, of the nucleic acid strands to be detected, to which one or more primers have hybridized, against the background of the nonhybridized primers by means of time-resolved fluorescence spectroscopy. The identification is preferably carried out by means of FCS, a measuring volume element of preferably from 0.1 to 20×10⁻¹⁵ 1 of the incubated solution being exposed to an excitation light of the laser, which excites the labeling groups present in said measuring volume so that they emit fluorescence light, the fluorescence light emitted from the measuring volume being measured by means of a photodetector and the change in the measured emission with time and the relative rate of diffusion of the molecules involved being correlated so that it is possible to identify at a correspondingly high dilution individual molecules in the measuring volume. It is possible to improve sensitivity by applying electric fields to the sample fluid, for example by capillary-electrophoretic separation of unbound labels and labels bound to analyte molecules, placing a capillary with an opening at the tip of <0.01 mm upstream of the measuring volume and generating in the capillary a constant electric field which moves the labels bound to the analyte in the direction of the measuring volume.

Although the method described in the German patent 195 08 366 has proved successful, there is a need, in particular when determining very low analyte concentrations, to further improve the sensitivity of the detection.

It was therefore the object of the present invention to provide a method for detecting an analyte at a low concentration in a sample fluid, which, on the one hand, avoids the disadvantages connected with amplification procedures and, on the other hand, has improved sensitivity.

This object is achieved by a method for directly detecting an analyte in a sample fluid, comprising the steps:

(a) contacting the sample fluid with one or more labeled analyte-specific receptors under conditions which enable the receptors to bind to the analyte, with an analyte-receptor complex which contains a greater number of labeling groups compared to receptors not bound to the analyte being formed in the presence of the analyte in the sample,

(b) passing the sample fluid or a portion thereof through a microchannel under conditions under which a predetermined flow profile exists in the microchannel, and

(c) identifying the analyte via the binding of receptor during flow through the microchannel.

The method of the invention makes possible the identification of analytes which are present in the sample fluid at extremely low concentrations of, for example, ≦10⁻⁹ mol/l and in particular ≦10⁻¹² mol/l. The sensitivity of the method is sufficiently high in order to be able to detect even analyte concentrations of down to 10⁻¹⁵ mol/l or 10⁻¹⁸ mol/l. The analytes are preferably biopolymers such as, for example, nucleic acids, peptides, proteins and protein aggregates, cells, subcellular particles, e.g. virions, etc. Particularly preferred analytes are nucleic acids, for example nucleic acids of pathogenic microorganisms, for example viral nucleic acids. The sample fluid is preferably a biological sample, for example a body fluid such as, for example, blood, urine, saliva, cerebrospinal fluid, lymph or a tissue extract.

The analyte is detected by binding of labeled analyte-specific receptors, resulting in the formation of an analyte-receptor complex which can be detected against the background of receptors not bound to the analyte. Suitable labeling groups are in particular non radioactive labeling groups and particularly preferably labeling groups detectable by optical methods, such as, for example, dyes and in particular fluorescence labeling groups. Examples of suitable fluorescence labeling groups are rhodamine, Texas Red, phycoerythrin, fluorescein and other fluorescent dyes common in diagnostic methods.

The labeled receptor is specific for the analyte to be detected, i.e. it binds to the analyte to be detected with sufficiently high affinity and selectivity under the assay conditions in order to make determination possible.

Examples of receptors preferably used for determining a nucleic acid analyte are labeled probes having a sequence complementary to the analyte and comprising oligonucleotides or nucleotide analogs, for example peptide nucleic acid (PNA). In a preferred embodiment, a plurality of different, preferably non overlapping labeled probes of preferably from 10 to 50 and particularly preferably from 15 to 20 nucleotide or nucleotide analog building blocks in length are used. In this connection, it is possible to use, for example, from 5 to 200, preferably 10 to 100 different probes in total, which can carry, where appropriate, different labeling groups which can, however, be detected together.

Labeled probes used as receptors can be added to the sample fluid in a prefabricated form. On the other hand, the labeled probes may also be generated in situ, i.e. in the sample fluid depending on the presence of the analyte. To this end, preferably unlabeled primers, labeled nucleotide building blocks and a corresponding nucleic acid polymerase, for example a DNA polymerase or a reverse transcriptase, are added to the sample fluid so that, in the presence of the analyte, the primer binds to said analyte and is extended enzymatically with incorporation of several labeled nucleotide building blocks. The labeled probe generated in situ in this way contains several labeling groups and can be distinguished from a nucleotide not incorporated into the probe, for example due to the higher fluorescence intensity.

It is also possible to determine other types of analytes, for example peptides, proteins and protein aggregates by using a plurality of different, preferably non competing labeled receptors, for example antibodies.

Advantageously, the labeled receptors are employed in a molar excess with respect to the analyte, preferably at a concentration of from 0.1 to 100 nM. Moreover, preference is given to the labeled receptors or, in the case of receptors generated in situ, the labeled receptor building blocks being different from analyte-receptor complexes with respect to physicochemical parameters such as molecular weight or/and charge so that it is possible to preconcentrate the analyte-receptor complexes by setting appropriate flow conditions.

A substantial feature of the method of the invention is passing the sample fluid or a portion thereof through a microchannel and identifying the analyte during the flow through the microchannel. Preferably, the flow is hydrodynamic but may also be electroosmotic in which case it is generated by an electric field gradient. A combination of hydrodynamic flow and field gradient is also possible. The flow through the microchannel preferably has a parabolic flow profile, i.e. the maximum flow rate is in the center of the microchannel and is reduced down to a minimum rate at the peripheries by way of a parabolic function. The maximum flow rate through the microchannel is preferably in the range from 1 to 50 mm/s, particularly preferably in the range from 5 to 10 mm/s. The diameter of the microchannel is preferably in the range from 1 to 100 μm, particularly preferably from 10 to 50 μm. The measurement is preferably carried out in a linear microchannel having an essentially constant diameter.

In addition, the analyte molecules may, where appropriate, be concentrated prior to the analyte determination in the microchannel by applying an electric field gradient. In a preferred embodiment of the invention, this electric field gradient is applied to a reaction chamber from which the analyte molecules are then directed into a microchannel. The reaction chamber may have a cylindrical or conical shape, for example the well of a microtiter plate. The electric field gradient can be generated by two electrodes in the reaction chamber, it being possible for one electrode to be arranged as a ring electrode concentrically around the upper part of the reaction chamber, while the second electrode may be arranged at the bottom of the reaction chamber as a point electrode or ring electrode with a smaller diameter. At the bottom of the reaction chamber, an orifice is located with the microchannel through which the particles preconcentrated in the electric field are passed and determined by suction or by applying pressure or by applying another electric field.

The analyte-receptor complex can be identified according to step c) of the method of the invention by means of any measurement method, for example by location-and/or time-resolved fluorescence spectroscopy, which measurement method is capable of recording very small signals of labeling groups, in particular fluorescence signals down to single-photon counting, in a very small volume element as is present in a microchannel. In this connection, it is important that the signals coming from unbound receptors or receptor building blocks are distinctly different from those caused by the analyte-receptor complexes.

It is possible, for example, to carry out detection by means of fluorescence correlation spectroscopy for which a very small volume element, for example from 0.1 to 20×10⁻¹² 1 of the sample fluid flowing through the microchannel, is exposed to an excitation light of a laser, which excites the receptors present in said measuring volume so that they emit fluorescence light, the fluorescence light emitted from the measuring volume being measured by means of a photodetector and the change in the measured emission with time and the relative flow rate of the molecules involved being correlated so that it is possible to identify at a correspondingly high dilution individual molecules in the measuring volume. For details of carrying out the method and details of the apparatuses used for detection, reference is made to the disclosure of the European patent 0 679 251.

Alternatively, detection may also be carried out via a time-resolved decay measurement, so-called time gating, as described, for example, by Rigler et al., “Picosecond Single Photon Fluorescence Spectroscopy of Nucleic Acids”, in: “Ultrafast Phenomenes”, D. H. Auston, Ed., Springer 1984. In this case, the fluorescent molecules are excited in a measuring volume and, subsequently, preferably at a time interval of ≧100 ps, a detection interval on the photodetector is opened. In this way it is possible to keep background signals generated by Raman effects sufficiently low so as to make possible an essentially interference-free detection. Time gating is particularly suitable for measuring quenching or energy transfer processes.

The detection is carried out under conditions which make it possible to discriminate between analyte-bound receptors and receptors not bound to the analyte. This discrimination of analyte-receptor complexes and unbound receptor molecules is due to the fact that the complex contains a multiplicity of labeling groups, while an unbound receptor or, in the case of a receptor generated in situ, a receptor building block has only a considerably smaller number of labeling groups, usually only a single labeling group. This difference in the fluorescence intensities of analyte-receptor complex and unbound receptor make it possible to set a cut-off in the detector, i.e. the detector is set in such a way that it registers the presence of just a single labeling group in the detection area only as background noise, while recognizing the greater number of labeling groups in the analyte-receptor complex as a positive signal.

Increasing the detection probability of analyte-receptor complexes, which is essential for the invention, and thus improving the sensitivity is achieved by setting the predetermined flow profile in the microchannel and, where appropriate, by suitable preconcentration measures. Owing to,—for example by the different molecular weight or/and different charge—of the complex of analyte molecule and receptor(s) compared with the usually smaller unbound receptors or, in the case of receptors generated in situ, the smaller receptor building blocks, there are differences in the migration behavior through the electric field or/and the microchannel which result in the analyte-receptor complexes being concentrated by at least a factor of 10⁴ compared to the untreated sample fluid.

The invention further relates to an apparatus for directly detecting an analyte in a sample fluid, comprising:

(a) a reaction chamber for contacting the sample fluid with one or more labeled receptors, with an analyte-receptor complex which contains a greater number of labeling groups compared to receptors not bound to the analyte being formed in the presence of the analyte in the sample,

(b) means for introducing sample fluid and receptors into the reaction chamber,

(c) a microchannel through which the sample fluid or a portion thereof can be passed using a predetermined flow profile,

(d) means for identifying analyte-receptor complexes during flow through the microchannel.

The apparatus preferably contains devices for automatic manipulation, heating or cooling devices such as Peltier elements, reservoirs and, where appropriate, supply lines for sample fluid and reagents and also electronic devices for analysis.

The method of the invention and the apparatus of the invention can be employed for all diagnostic methods for direct detection of analytes.

Further features of the invention are stated in claims 22 to 26.

Furthermore, the present invention is to be illustrated by the following figures in which:

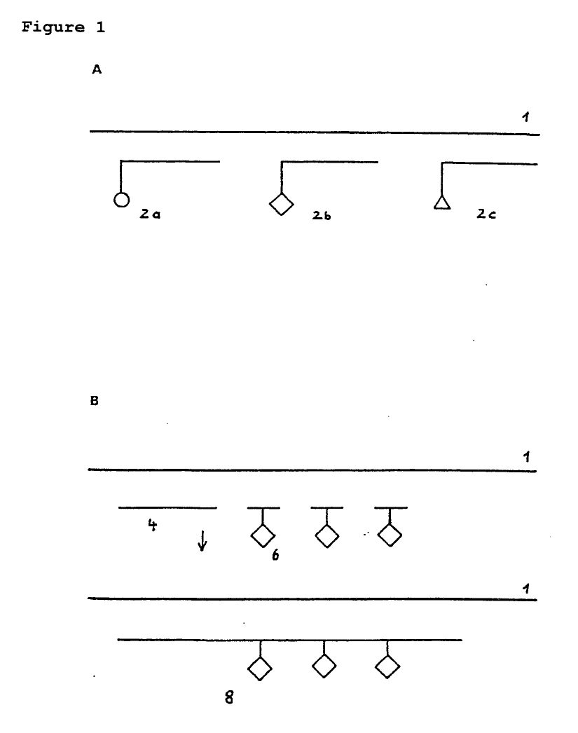

FIG. 1 shows two embodiments for carrying out the method of the invention. (A): The analyte (1), for example a nucleic acid molecule such as, for example, a viral DNA, is contacted with a multiplicity of receptor probes (2a, 2b, 2c) which carry identical or different labeling groups and can, at the same time, bind to the analyte. (B): The nucleic acid analyte is contacted with a primer (4) complementary thereto, labeled nucleotide building blocks (6) and an enzyme (not shown) suitable for primer extension. Enzymatic primer extension generates an extended receptor molecule which carries several labeling groups and is complementary to the analyte. Both embodiments have in common that the analyte-receptor complex which forms in the presence of the analyte has a greater number of labeling groups than the receptor molecules or receptor building blocks which are present in the absence of the analyte.

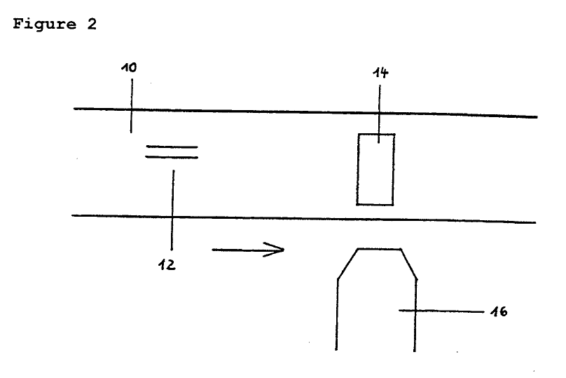

FIG. 2 shows the diagrammatic representation of detecting analyte-receptor complexes in a microchannel. The analyte-receptor complexes (12) migrate in a microchannel (10) having a predetermined flow profile to a detecting volume (14). In the detecting volume (14), detection is carried out by means of a detector (16). The detector may comprise, for example, an apparatus for fluorescence correlation spectroscopy, which has a laser which illuminates the detecting volume via a beam splitter and confocal imaging optics and projects it onto a photodetector.

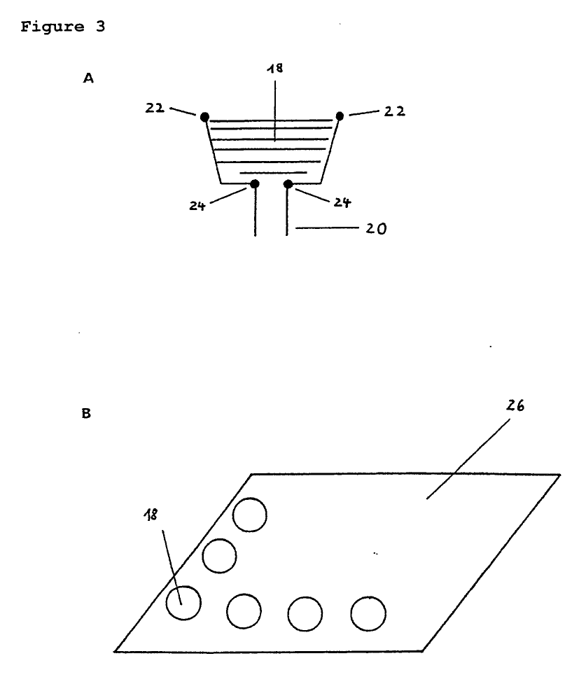

FIG. 3 shows the diagrammatic representation of a preferred apparatus for carrying out the method of the invention. (A): The apparatus contains a reaction chamber (18) in which sample fluid and receptor molecules can be contacted with one another and then passed on into the microchannel (20) for detection, as shown in FIG. 2, using pressure or suction or by applying another electric field gradient. Preconcentration is carried out in the reaction chamber by applying an electric field gradient between the electrodes (22) and (24). The electrode (22), usually the anode, may be in the form of a ring around the upper region of the reaction chamber (18). The electrode (24), usually the cathode, is located at the bottom of the reaction chamber and may be, for example, in the form of a metal layer and, where appropriate, likewise in the form of a ring. (B): The apparatus (26) may contain a multiplicity of reaction chambers (18) as depicted in FIG. 3 (A), in order to enable parallel processing of a multiplicity of samples, for example 10 to 100 samples.

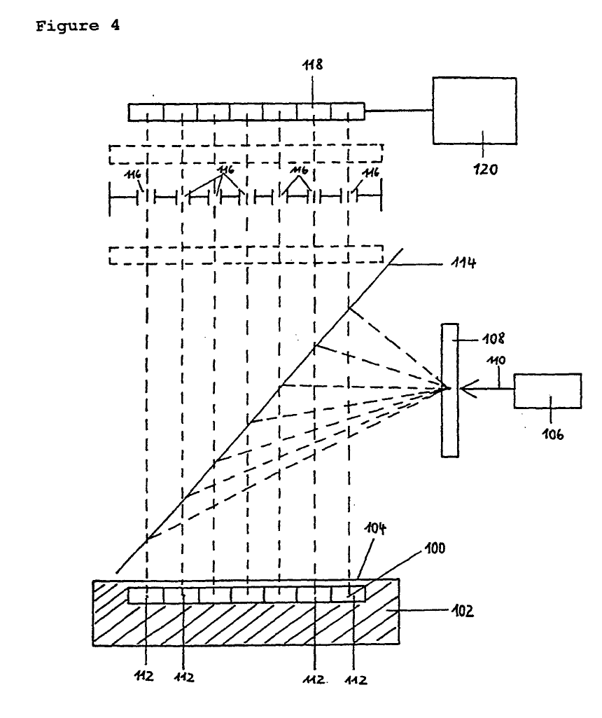

FIG. 4 depicts a diagrammatic and greatly simplified representation of another embodiment of an apparatus for detecting fluorescent molecules, in particular individual molecules, in a sample fluid flowing through a microchannel.

According to FIG. 4, the microchannel 100 (depicted as running perpendicularly to the plane of the drawing) is designed inside a support 102 which is translucent on the side 104 toward the microchannel 100, at least for the wavelengths of the fluorescence excitation light, which are of interest here, and for the wavelengths of the fluorescence light.

The apparatus according to FIG. 4 comprises a laser 106 as a light source, in whose light path an optical diffraction element or a phase-modulating element 108 is arranged, which generates from the laser beam 110 via light diffraction a diffraction pattern in the form of a linear or two-dimensional array of “focal points” 112. The diffracted and phase-modulated beams extending from the diffraction element 108 are reflected toward the microchannel 100 by a dichroic and, respectively, wavelength-selective mirror 114, the arrangement preferably being made in such a way that the focal points (also referred to as confocal volume elements 112 hereinbelow) form an essentially unbroken “detecting curtain” across the cross section of the microchannel 100. Each molecule migrating through the microchannel 100 in a sample solution in question must thus pass the “detecting curtain”, i.e. at least one of the confocal volume elements 112. If the molecule in question is made to fluoresce by the laser light, then the presence of such a molecule can be detected by recording and analyzing the fluorescence light.

The fluorescence light can pass the dichroic mirror toward the top of FIG. 4.

According to FIG. 4, pinhole apertures 116 are provided in relation to the confocal volume elements 112 at sites which are in each case paired with the confocal volume elements 112. A photodetector arrangement 118 which may be a group of individual avalanche photodetectors (avalanche diodes) or may be avalanche photodetectors integrated in a matrix (array) on a chip is located in the optical light path behind the pinhole apertures 116. A controling device or analyzing device 120 analyzes the output signals of the photodetector arrangement 118. The analyzing unit 120 contains means for correlating the signals so that the apparatus 4 for carrying out fluorescence correlation spectroscopy as is explained in principle, for example, in Bioimaging 5 (1997) 139-152 “Techniques for Single Molecule Sequencing”, Klaus Dörre et al. [lacuna].

In the apparatus according to FIG. 4, a confocal projection of the measuring volumes or confocal volume elements 112 takes place onto the relevant photodetector elements of the arrangement 118. Fluorescence light which emits from one or, where appropriate, several molecules which have been made to fluoresce in the relevant volume elements 112 by the laser light is projected via the dichroic mirror 114 into the pinhole apertures 116 paired with the relevant focal volume elements 112 and finally onto the assigned element of the detector arrangement 118. In FIG. 4, 120 and 122 refer to diagrammatically depicted imaging elements. The analyzing unit 120 which may be, for example, a personal computer containing a correlation card analyzes the output signals of the detector arrangement 118 in order to be able to provide information about the presence of particular fluorescent molecules, in particular individual molecules.

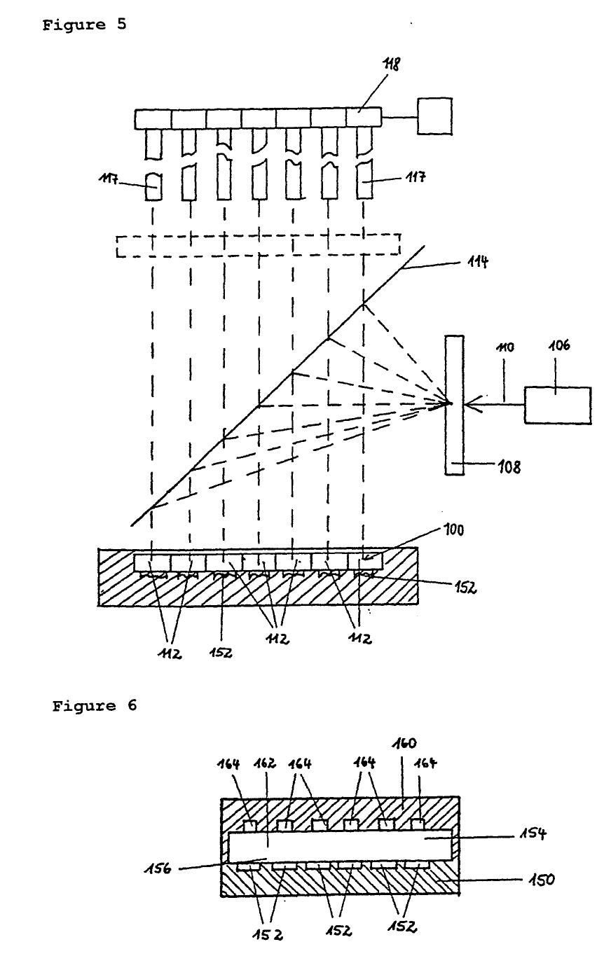

FIG. 5 depicts a modification of the apparatus of FIG. 4.

Instead of the pinhole aperture arrangement 116 of FIG. 4, the arrangement according to FIG. 5 has a correspondingly arranged array of light-guiding fibers (glass fiber bundles) 117 whose light-entry areas are located at the sites paired with the assigned confocal volume elements 112. The light-guiding fibers are optically connected to a photodetector arrangement 118 which may correspond to the photodetector arrangement 118 of FIG. 4. Otherwise, the apparatus according to FIG. 5 corresponds to the apparatus according to FIG. 4. Both apparatuses are suitable for carrying out the method as claimed in any of claims 1-19 and, very generally, for carrying out methods concerned with the detection of molecules in highly diluted sample solutions, in particular with the detection of individual molecules, for example in the sequencing of nucleic acids.

It should further be noted that the photodetector elements need not necessarily be avalanche diodes but that other detectors, for example photomultipliers, CCD sensors, etc. may also be used as alternatives.

FIG. 6 depicts another embodiment of a detecting apparatus according to the invention for detecting molecules, in particular individual molecules, in highly diluted sample solutions. The arrangement according to FIG. 6 comprises a substrate or a support 150 having a linear or two-dimensional array of surface-emitting lasers, in particular quantum well lasers 152, which emit light into the microchannel 154 at the area 156 which forms the boundary of the microchannel 154. The microchannel 154 extends perpendicularly to the plane of the drawing in FIG. 6. Owing to its radiation characteristics, each laser element 152 covers a particular volume area of the microchannel 154 with its radiation field. The volume elements illuminated by the laser elements 152 should be located side by side so closely or should, where appropriate, overlap each other that they form in their entirety a “detecting curtain” as unbroken as possible in the sense that each analyte molecule can pass the microchannel 154 only by passing through a relevant volume element.

In the substrate region or support region 160, photodetectors 164 are grouped on the channel boundary wall 162 opposite the area 156 to give an array which corresponds geometrically essentially to the array of laser elements 152 so that a particular photodetector element 164 is assigned opposite a particular laser element 152. The photodetectors 164 are preferably integrated avalanche photodiodes.

The elements described so far with reference to FIG. 6 preferably form components of an integrated chip component with connectors (not shown) for the energy supply and control of the laser elements 152 and for the energy supply and signal output of the photodetector elements 164.

The signals obtained from the photodetectors 164 can be analyzed by means of an analyzing unit connected to the chip component, the analyzing unit preferably comprising a correlation device so that the arrangement shown in FIG. 6 is suitable for fluorescence correlation spectroscopy (FCS).

The laser elements 152 form the excitation light sources for fluorescence excitation of the molecules capable of fluorescence which flow through the microchannel 154. The photodetector elements 164 are sensitive to light of the relevant fluorescence wavelength or fluorescence wavelengths. The arrangement may, where appropriate, contain spectral filters to provide the detector elements 164 with wavelength selectivity.

The arrangement according to FIG. 6 can be used for carrying out the method as claimed in any of claims 1-19 and, in addition and very generally, for carrying out methods concerned with the detection of molecules in highly diluted sample solutions, in particular of individual molecules.

Mention should also be made of a possible modification of the apparatuses according to FIG. 4 and FIG. 5. This modification consists of providing for, in a similar manner as for the channel 154 in FIG. 6, laser elements 152 as are indicated in dashes in FIG. 5 also directly on the channel 100. These may be, for example, quantum well laser elements which are integrated in a substrate 102 containing the channel 100. The elements 106 and 108 can then be dispensed with.

It should be noted that the apparatuses according to FIGS. 4-6 and the modifications mentioned have, where appropriate, an independent meaning within the scope of-the invention.

1. A method for directly detecting an analyte in a sample fluid, comprising the steps:
(a) contacting the sample fluid with one or more labeled analyte-specific receptors under conditions which enable the receptors to bind to the analyte, with an analyte-receptor complex which contains a greater number of labeling groups compared to receptors not bound to the analyte being formed in the presence of the analyte in the sample,
(b) passing the sample fluid or a portion thereof through a microchannel under conditions under which a predetermined flow profile exists in the microchannel, the flow being a hydrodynamic flow, and
(c) identifying the analyte-receptor complex during flow through the microchannel.

2. The method as claimed in claim 1,
characterized in that
the analyte is selected from the group consisting of nucleic acids, peptides, proteins and protein aggregates.

3. The method as claimed in either of claims 1 and 2,
characterized in that
the analyte concentration in the sample fluid is ≦10⁻⁹ mol/l and in particular ≦10⁻¹² mol/l.

4. The method as claimed in any of claims 1 to 3,
characterized in that
the receptors used for determining a nucleic acid analyte are labeled probes having a sequence complementary to said analyte.

5. The method as claimed in claim 4,
characterized in that
a plurality of different, preferably non overlapping, labeled probes are used.

6. The method as claimed in either of claims 4 and 5,
characterized in that
the labeled probes are added to the sample fluid in a prefabricated form.

7. The method as claimed in either of claims 4 and 5,
characterized in that
the labeled probes are generated in situ by adding primers, labeled nucleotide building blocks and a nucleic acid polymerase to the sample fluid and extending the primers enzymatically in the presence of the analyte with incorporation of the labeled nucleotide building blocks.

8. The method as claimed in any of claims 1 to 3,
characterized in that
the receptors used for determining an analyte selected from the group consisting of peptides, proteins and protein aggregates are labeled antibodies against said analyte.

9. The method as claimed in any of the preceding claims,
characterized in that
the labeled receptors are employed in a molar excess with respect to the analyte.

10. The method as claimed in any of the preceding claims,
characterized in that
the labeling groups are dyes, in particular fluorescent dyes.

11. The method as claimed in any of the preceding claims,
characterized in that
the flow has a parabolic flow profile.

12. The method as claimed in any of the preceding claims,
characterized in that
the diameter of the microchannel is in the range from 1 to 100 μm.

13. The method as claimed in any of the preceding claims,
characterized in that
the maximum flow rate through the microchannel is in the range from 1 to 50 mm/s.

14. The method as claimed in any of the preceding claims,
characterized in that
the analyte molecules are additionally concentrated in an electric field.

15. The method as claimed in claim 14,
characterized in that
the electric field is applied to a reaction chamber from which the analyte molecules are directed into a microchannel.

16. The method as claimed in claim 15,
characterized in that
the reaction chamber has a cylindrical or conical shape.

17. The method as claimed in any of the preceding claims,
characterized in that
the analyte is identified using fluorescence correlation spectroscopy.

18. The method as claimed in any of the preceding claims,
characterized in that
the measurement is carried out in one or more confocal spatial elements or/and by time gating.

19. An apparatus for directly detecting an analyte in a sample fluid, comprising:
(a) a reaction chamber for contacting the sample fluid with one or more labeled receptors, with an analyte-receptor complex which contains a greater number of labeling groups compared to receptors not bound to the analyte being formed in the presence of the analyte in the sample,
(b) means for introducing sample fluid and receptors into the reaction chamber,
(c) a microchannel through which the sample fluid or a portion thereof can be passed using a predetermined flow profile, the flow being a hydrodynamic flow, and
(d) means for identifying analyte-receptor complexes during flow through the microchannel.

20. The use of the apparatus as claimed in claim 19 for carrying out said method as claimed in any of claims 1 to 18.

21. An apparatus for detecting fluorescent molecules in a sample fluid flowing through a microchannel with a hydrodynamic flow, having
a laser (106) as fluorescence excitation light source for said molecules,
an optical arrangement (114, 116, 120, 122) for guiding and focusing laser light of the laser (106) to a focal area of the microchannel (100) and for confocally projecting the focal area onto a photodetector arrangement (118) to record fluorescence light which has been emitted in the focal area by one or, where appropriate, more optically excited molecules,
characterized in that
the optical arrangement has a diffracting element (108) or a phase-modulating element (108) in the light path of the laser (106), which element, where appropriate in combination with one or more optical imaging elements, is set up to generate from the laser beam of the laser (106) a diffraction pattern in the form of a linear or two-dimensional array of focal areas (112) in the microchannel, the optical arrangement being set up to project each focal area (112) confocally for fluorescence detection by the photodetector arrangement (118).

22. The apparatus as claimed in claim 21, characterized in that the photodetector arrangement (118) is connected to an analyzing device (120) which has a correlating device for the fluorescence correlation-spectroscopic analysis of the photodetector signals.

23. An apparatus for detecting fluorescent molecules in a sample fluid flowing through a microchannel (154), characterized by two walls (156, 162) which mark the boundary of the microchannel 154 on opposite sides and one of which has an array of preferably integrated laser elements (152) emitting into the microchannel (154) as fluorescence excitation light sources and the other one of which has an array of preferably integrated photodetector elements (164), arranged in each case opposite the laser elements (152), as fluorescence light detectors.

24. The apparatus as claimed in claim 23, characterized in that the laser elements (152) are quantum well laser elements.

25. The apparatus as claimed in either of claims 23 and 24, characterized in that the photodetector elements (164) are avalanche diodes.
